(19) United States
(12) Patent Application Publication
(10) Pub. No.: US 2023/0381749 A1
(43) Pub. Date: Nov. 30, 2023

(54) DEHYDROGENATION CATALYST FOR PREPARING OLEFIN FROM ALKANE GAS AND METHOD FOR PRODUCING SAME

(71) Applicants: SK GAS CO., LTD., Seongnam-si, Gyeonggi-do (KR); KOREA RESEARCH INSTITUTE OF CHEMICAL TECHNOLOGY, Daejeon (KR)

(72) Inventors: Daesung PARK, Daejeon (KR); Hawon PARK, Daejeon (KR); Changyeol SONG, Daejeon (KR); Yong Ki PARK, Seoul (KR); Won Choon CHOI, Daejeon (KR); Ung Gi HONG, Seongnam-si, Gyeonggi-do (KR); Deuk Soo PARK, Seongnam-si, Gyeonggi-do (KR); Miyoung LEE, Seongnam-si, Gyeonggi-do (KR); Hae Bin SHIN, Seongnam-si, Gyeonggi-do (KR); Sanghyeon PARK, Seongnam-si, Gyeonggi-do (KR)

(73) Assignees: SK GAS CO., LTD., Seongnam-si, Gyeonggi-do (KR); KOREA RESEARCH INSTITUTE OF CHEMICAL TECHNOLOGY, Daejeon (KR)

(21) Appl. No.: 18/032,990

(22) PCT Filed: Nov. 1, 2021
(86) PCT No.: PCT/KR2021/015488

§ 371 Date: Apr. 20, 2023

(30) Foreign Application Priority Data
Nov. 3, 2020 (KR) 10-2020-0144864

(51) Int. Cl. B01J 23/75; B01J 35/00; B01J 35/02; B01J 21/04; B01J 23/42; B01J 37/02; B01J 6/00; C07C 5/333

(52) U.S. Cl. CPC B01J 23/75; B01J 35/0006; B01J 35/02; B01J 21/04; B01J 23/42; B01J 37/0211; B01J 37/0209; B01J 6/001; C07C 5/3337

(57) ABSTRACT

There is provided a dehydrogenation catalyst for producing olefins from alkane gases, in which a metal active component is supported on an alumina carrier containing boron. There is provided a method for preparing a dehydrogenation catalyst for producing olefins from alkane gases. The method includes impregnating alumina in a boron-containing solution and calcining it to provide a boron-alumina carrier; providing a solution containing the metal active component; impregnating the boron-alumina solution in the solution containing the metal active component and drying it; and calcining the boron-alumina carrier on which the metal active component is supported at 700° C. to 900° C.

### CROSS-REFERENCE TO PRIOR APPLICATIONS

This application is a National Stage patent Application of PCT International Patent Application No. PCT/KR2021/015488 (filed on Nov. 1, 2021) under 35 U.S.C. § 371, which claims priority to Korean Patent Application No. 10-2020-0144864 (filed on Nov. 3, 2020), which are all hereby incorporated by reference in their entirety.

### BACKGROUND

The present invention relates to a catalyst for producing olefins with improved selectivity and conversion rate compared to the conventional art in the production of olefins from alkane gases such as ethane, propane, and butane, and a method for preparing the same.

Olefins such as ethylene and propylene are widely used in the petrochemical industry. In general, these olefins are obtained in the naphtha thermal cracking process. However, as shale gas production surged and the price competitiveness of gas raw materials improved compared to naphtha, the ethane thermal cracking process increased rapidly. As a result, ethylene supply increased, and as propylene production relatively slowed, an imbalance between propylene supply and demand was exhibited. Therefore, “on purpose propylene” for controlling the supply and demand of propylene—special purpose propylene manufacturing technology is becoming widespread, and propylene production through the dehydrogenation process of lower hydrocarbons using a catalyst is required as an important technology.

The existing propane dehydrogenation (PDH) commercial process typically includes a fixed-bed reactor and a moving-bed reactor.

In contrast, there have been no commercialization cases in PDH technology (FPDH, fast-fluidized propane dehydrogenation) using a high-speed fluidized bed (hereinafter referred to as a fluidized bed) reactor until now.

The biggest difference between the fixed-bed reactor and the fluidized bed reactor is a contact time between the catalyst and the reactant (propane). In other words, the fluidized bed reactor is a process in which propane and a catalyst are injected together into the fluidized bed reactor at a very high rate to perform the reaction, and then the catalyst goes into the regeneration unit and the product goes into the separation unit.

The goal of the FPDH process that has conventionally been developed is to set the residence time of the catalyst to 10 seconds or less. If the residence time of the catalyst is short, the injection rate of the supply amount of propane is also fast, and the catalyst is immediately regenerated and participates in the reaction again. Therefore, when the FPDH process is developed as a commercial process, propylene output is greatly increased compared to the fixed bed process.

However, since the contact time between the catalyst and propane is short, the efficiency of the catalyst becomes very important. In other words, it is important to maximize the selectivity and conversion rate, which are two efficiency measures of the catalyst, respectively.

Furthermore, since the propane dehydrogenation process technologies that are currently being used are configured based on noble metal catalysts or a non-continuous process, they are experiencing difficulties in propylene production operation such as a reactor clogging phenomenon due to excessive activity (coke production) of noble metal catalysts, fixed-bed reactor valve sequence troubles, and the like.

In addition, the propane dehydrogenation reaction has a thermodynamical limitation in the propane conversion rate due to a reversible reaction by hydrogen. In order to overcome such a problem, most processes use external oxidizers such as oxygen, halogen, sulfur compounds, carbon dioxide, water vapor, and the like to convert hydrogen into water.

Therefore, for effective mass production of propylene, it is required to develop a new propane dehydrogenation process that solves the problems of the continuous process, and reduces production cost by using a direct-type dehydrogenation catalyst without an oxidizer.

Among the catalysts used for propane dehydrogenation, the reaction proceeds with a direct dehydrogenation mechanism in which hydrogen is adsorbed on the active site in the case of noble metal catalysts, but the mechanism has not been clearly identified due to the incompleteness of the active site due to the mobility of electrons in the case of transition metal oxides.

Under these circumstances, the most commonly used catalysts as usual PDH catalysts are Pt—Sn, VOₓ, and CrOₓ catalysts. Although the CrOₓ catalyst is very excellent in terms of propane conversion and selectivity, its use is limited due to problems such as environmental pollution and human harm, and difficulties in controlling the oxidation reaction in the initial stage of the reaction. The platinum catalyst has excellent selectivity, but it is expensive, and the rate of coke production is very fast so that detailed control thereof is required. In addition, the specific activity of the catalyst varies depending on the combination of Sn, which is a cocatalyst component, and other metals, and also, the development of a new multi-component catalyst is continuously required for platinum catalysts due to the increase in environmental hazards of Sn.

Meanwhile, in the case of conventional catalysts including the catalysts of Patent Documents 1 and 2, deactivation becomes a problem due to coke deposition of the catalyst.

Accordingly, the present inventors have developed a catalyst for producing olefins, which is excellent in terms of conversion rate and selectivity of the catalyst at the same time compared to the conventional art by introducing a new catalyst through continuous research, and a method for preparing the same.

(Patent Document 1) Korean Patent Publication No. 2018-0079178

(Patent Document 2) International Publication Number WO2016/13561

### SUMMARY

An object of the present invention is to provide a catalyst for producing olefins, which has excellent conversion rate and selectivity in the production of olefins from alkane gases such as ethane, propane, and butane, and a method for preparing the same.

In a catalyst for producing olefins from alkane gases according to the present invention, a metal active component is preferably supported on an alumina carrier containing boron.

The boron is preferably supported in an amount of 0.1 to 2 wt % based on the alumina.

The boron is more preferably supported in an amount of 0.5 to 2 wt % based on the carrier.

Preferably, the metal active component essentially includes cobalt.

The cobalt is preferably supported in an amount of 1 to 5 wt % based on the alumina.

More preferably, the metal active component further includes platinum.

The platinum is preferably supported in an amount of 0.001 to 0.05 wt % based on the alumina.

It is preferable that a method for preparing a catalyst for producing olefins from alkane gases according to the present invention include the steps of:
impregnating alumina in a boron-containing solution and calcining it to provide a boron-alumina carrier;
providing a solution containing the metal active component;
impregnating the boron-alumina solution in the solution containing the metal active component and drying it; and
calcining the boron-alumina carrier on which the metal active component is supported at 700° C. to 900° C.

The boron-alumina carrier is preferably calcined at 400 to 600° C.

Another aspect of the present invention is to provide a continuous reaction-regeneration olefin production method including a catalyst for producing olefins from alkane gases prepared according to the present invention.

In the continuous reaction-regeneration olefin production method, the reaction temperature is preferably 560 to 640° C.

In the continuous reaction-regeneration olefin production method, it is preferable that alkane, which is a raw material, has a flow rate (weight hour space velocity) of 4 to 16 h⁻¹.

Although the catalyst for producing olefins from alkane gases such as ethane, propane, and butane according to the present invention and the method for preparing the same are excellent in terms of conversion rate and selectivity so that they are effective in both fixed bed reactors and fluidized bed reactors, the realization of FPDH processes, which have not been conventionally commercially realized, particularly is enabled. In particular, the catalyst according to the present invention has high conversion rate and selectivity by significantly improving a catalyst deactivation phenomenon caused by coke deposition compared to conventional catalysts.

### BRIEF DESCRIPTION OF THE DRAWINGS

FIG. 1 schematically shows activity results of the initial stage (1 to 3 seconds) of the PDH reaction of commercial alumina according to the boron content.

FIG. 2 schematically shows comparison results of the PDH activity test of 4Co/Al₂O₃ and 4Co-0.7B/Al₂O₃ catalysts.

FIG. 3 schematically shows results of the PDH reaction activity test of a 4Co-0.01Pt/Al₂O₃ catalyst according to the boron content.

FIG. 4 schematically shows catalyst images after 1 minute PDH reaction of a 4Co-0.01Pt-x % B catalyst.

FIG. 5 schematically shows comparison results of the PDH initial activity test of a Co—Pt catalyst and a Co—Pt—B catalyst.

FIG. 6 schematically shows TOS activity test results in the initial stage of PDH of a Co—Pt—B catalyst according to the boron content.

FIG. 7 schematically shows results of continuous reaction-regeneration and recycling reaction activities of a 4Co-0.01Pt/Al₂O₃ catalyst and a 4Co-0.01Pt-0.7B/Al₂O₃ catalyst.

FIG. 8 schematically shows detailed results of continuous reaction-regeneration and recycling reaction activities of a 4Co-0.01Pt-0.7B/Al₂O₃ catalyst.

### DETAILED DESCRIPTION

In the catalyst for producing olefins from alkane gases according to the present invention, the metal active component is preferably supported on an alumina carrier containing boron.

Hereinafter, preferred embodiments of the present invention will be described with reference to the accompanying drawings. However, the embodiments of the present invention may be modified in various different forms, and the scope of the present invention is not limited to the embodiments described below.

In describing the present embodiments, the same names and reference numerals are used for the same components, and thus overlapping additional descriptions are omitted below. Scale ratios are not applied in the drawings referred to below.

In the catalyst for producing olefins from alkane gases according to the present invention, the metal active component is preferably supported on an alumina carrier containing boron.

The alumina carrier preferably has a γ to θ phase at a production temperature of 550 to 850° C. that is the dehydrogenation reaction temperature or higher, and has a surface area of 80 to 300 m²/g in this range.

When the carrier is prepared at a temperature lower than the dehydrogenation reaction temperature, thermal deformation of the catalyst may occur during the dehydrogenation reaction, and when it is prepared at a temperature exceeding 900° C., the catalyst has a low surface area due to crystallization of the carrier, which may impede the mass transfer for catalytic activity when the catalyst is in contact with the reactants.

Since coke deposition resulting from the acid point of alumina, a support commonly used for catalysts, is dominant, the initial reaction activity of the PDH reaction was tested by first changing the amount of boron to 0 to 2 wt % and supporting it on Puralox, which is a commercial alumina carrier.

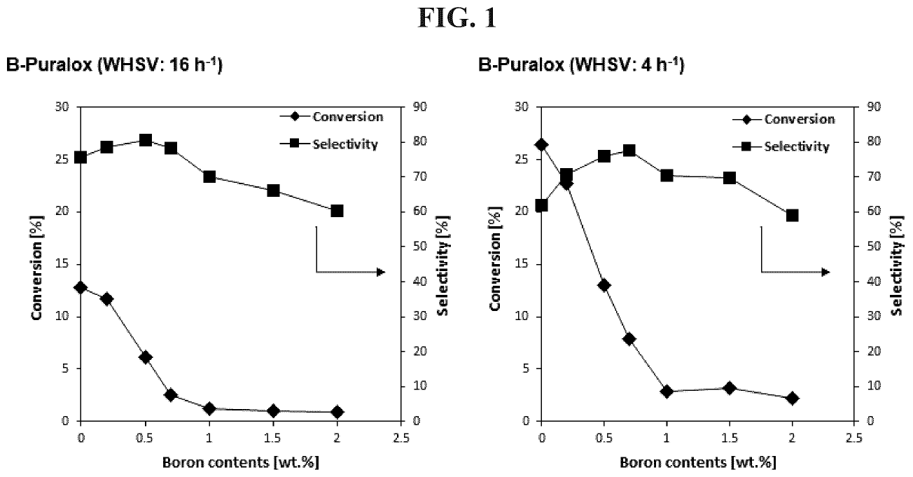

Tests were conducted in a lab-scale fixed bed reactor, and the average values of TOS between 1 and 3 seconds of the initial stage of the reaction, which are the results of the tests, are shown in FIG. 1. Reaction tests were conducted under conditions of 600° C., WHSV 16 h⁻¹, and WHSV 4 h⁻¹.

Under the condition of flow rate WHSV 16 h⁻¹ only with alumina without Co or Pt metal, which is the main active site, a conversion rate of 10% or more was shown at the beginning of the reaction. In addition, under the flow rate WHSV 4 h⁻¹ condition, which is 4 times slower, the conversion rate was shown to be 25% or more, and the production rate of methane, ethylene, or the like was high due to cracking, that is a side reaction rather than propylene production.

Thereafter, it was confirmed that the activity of alumina rapidly dropped as the amount of boron supported on alumina increased from 0.2 wt % to 1 wt %. When the supporting amount of boron was further increased from 1 wt % to 2 wt %, the reduction in conversion rate became insignificant, and a trend of decreasing the selectivity was shown. This means that boron can effectively suppress side reaction sites of alumina, thereby suppressing coke deposition and by-product production. Therefore, the content of boron is determined to be most effective at 0.5 to 2 wt % based on the alumina.

Traditionally, active metals for dehydrogenation catalysts are diverse, but cobalt is preferable in order to obtain high selectivity at the very beginning of the reaction within a few seconds, which is a characteristic of the FPDH process. Furthermore, platinum is preferably added in order to improve the conversion rate while maintaining the high selectivity properties of cobalt-based catalysts.

The cobalt is preferably supported in an amount of 1 to 5 wt % based on the alumina. A catalyst amount outside the above range is outside the commercially applicable range for FPDH.

In addition, since crystalline oxide is formed when the catalyst amount is large, it is not preferable as a dehydrogenation catalyst. Furthermore, when the catalyst amount is increased beyond the above range, the yield is significantly reduced.

In the reaction within a TOS of 1 to 3 seconds of the propane dehydrogenation reaction, the cobalt catalyst shows the highest selectivity, and platinum seems to contribute the most to the conversion rate. Therefore, it is assumed that the platinum catalyst compensates for the low conversion rate of the cobalt catalyst having high selectivity.

It can be seen that as the amount of platinum increases, the total propylene yield also increases while the propane conversion rate increases. However, as the amount of platinum increased, side reactions also continuously increased, and the main by-products were methane and ethane. This indicates that the platinum catalyst has a very high activity not only in the dehydrogenation reaction but also in the hydrogenolysis reaction in which generated hydrogen and propane meet to form methane and ethane.

Therefore, considering the increase section in conversion rate and the continuous decrease in selectivity according to the introduction amount of platinum, it can be seen that the platinum supported in an amount of 0.001 to 0.05 wt % based on the alumina is the most suitable catalyst for application to a fast circulating fluidized bed process.

Meanwhile, it is preferable that the method for preparing a catalyst for producing olefins from alkane gases according to the present invention include the steps of:
impregnating alumina in a boron-containing solution and calcining it to provide a boron-alumina carrier;
providing a solution containing the metal active component;
impregnating the boron-alumina solution in the solution containing the metal active component and drying it; and
calcining the boron-alumina carrier on which the metal active component is supported at 700° C. to 900° C.

The catalyst is preferably calcined at 700° C. to 900° C. The catalyst phase changes depending on the calcination temperature of the catalyst, but it is not preferable as a dehydrogenation catalyst since it mainly causes oxidation-reduction reactions since it forms a nano-sized crystal phase outside the above temperature range.

The boron-alumina carrier is preferably calcined at 400 to 600° C. In order to use it as a carrier, it is preferable to maintain a large specific surface area, and when the temperature is higher than the above range, the phase of the alumina carrier changes so that the surface area may decrease, and crystallization may proceed.

Conventionally, catalysts synthesized by the sol-gel method and the precipitation method, which are expected to have high crystallinity, are not preferable since CO₂ is mainly produced by oxidation reaction rather than dehydrogenation reaction. Meanwhile, in the case of a medium pore catalyst by the EISA method that is a synthesis method in which the ratio of alumina is increased, or a catalyst synthesized by a precipitation method on an alumina solid slurry, the acid point of the alumina support may be appropriately controlled and increase the selectivity of the dehydrogenation reaction.

Another aspect of the present invention is to provide a continuous reaction-regeneration olefin production method including a catalyst for producing olefins from alkane gases prepared according to the present invention. More preferably, it is to produce propylene from propane.

In the continuous reaction-regeneration olefin production method, the reaction temperature is preferably 560 to 640° C. Since the dehydrogenation reaction (PDH) is an equilibrium reaction, a high reaction temperature is required. However, side reactions occur rapidly from 640° C. or higher, and at the same time, by-products increase due to thermal reactions (non-catalytic) caused by high temperatures. Therefore, in order to minimize the decrease in selectivity, a temperature the same as or higher than that is not desirable.

In addition, regeneration is required in order to remove coke deposition or the like during the reaction, and since the reaction temperature and the temperature of a regeneration part are interdependent, the regeneration part is set at a temperature approximately 20 to 30° C. higher than the reaction temperature. Therefore, in the case of a reaction at 560° C., coke is removed in the regeneration part at about 590° C. In a temperature range lower than this, there is a difficulty in regenerating the catalyst through rapid coke removal.

In the continuous reaction-regeneration olefin production method, it is preferable that the flow rate (WHSV) of alkane, which is a raw material, is 4 to 16 h⁻¹. More preferably, it is 12 to 16 h⁻¹. At a flow rate within the above range, the catalyst may be smoothly circulated and have a fast residence time (RT).

Hereinafter, the present invention will be described in more detail through preparation examples and examples.

### PRODUCTION EXAMPLES

1. Preparation of Boron Oxide-Alumina Carrier (B/Alumina)

Boric acid was used as a boron precursor for preparing a boron oxide-alumina carrier.

In order to prepare a metal oxide solution, methanol was prepared in an amount equal to the pore volume of alumina. A boron oxide solution was prepared by injecting H₃BO₃ (boric acid) having 0.2 to 2 wt % of boron (B) compared to alumina into prepared methanol and then stirring it for 2 to 4 hours.

The prepared metal oxide solution was added to alumina and impregnated by an incipient wetness impregnation method, and after the temperature was raised at a temperature raising rate of 2° C. per minute, it was calcined at 500° C. for 6 hours to prepare a boron oxide-alumina carrier.

2. Preparation of Cobalt/Boron Oxide-Alumina Catalysts Through Impregnation Method (Co/B-Alumina)

In order to prepare a metal oxide solution, water was prepared in an amount equal to the volume of the alumina pores. A solution was prepared by stirring Co(NO₃)₂·6H₂O (cobalt nitrate hexahydrate) containing 0 to 10 wt % of cobalt based on alumina in water.

The prepared metal oxide solution was added to the prepared boron oxide-alumina and impregnated by an incipient wetness impregnation method, and dried at 50 to 75° C. for 12 hours. Thereafter, cobalt/boron oxide-alumina catalysts were each prepared by raising the temperature at a temperature raising rate of 1° C. per minute and thus calcining it at 700° C. to 900° C. for 6 hours.

3. Preparation of Cobalt-Platinum/Boron Oxide-Alumina Catalysts Through Co-Impregnation Method

(Co—Pt/B-Alumina)

In order to prepare a metal oxide solution, water was prepared in an amount equal to the volume of the alumina pores. A cobalt-platinum oxide solution was prepared by co-impregnating Co(NO₃)₂·6H₂O (cobalt nitrate hexahydrate) containing 0 to 10 wt % of cobalt and H₂PtCl₆.xH₂O (chloroplatinic acid) containing 0 to 200 ppm (0 to 0.02 wt %) of platinum based on alumina.

The prepared metal oxide solution was added to the prepared boron oxide-alumina and impregnated by an incipient wetness impregnation method, and dried at 50 to 75° C. for 12 hours. Thereafter, cobalt-platinum/boron oxide-alumina catalysts were each prepared by raising the temperature at a temperature raising rate of 1° C. per minute and thus calcining it at temperature of 700° C. to 900° C. for 6 hours.

<Continuous Reaction Regeneration Test Method (Recycle Test) and Activity Evaluation>

After injecting the prepared catalysts into a fixed-bed type reactor using an automatic continuous reaction system equipped for continuous reaction regeneration, the reactor reached 600° C., which is a reaction and regeneration temperature, in an atmosphere of nitrogen gas that is an inert gas at a temperature raising rate of 10° C. per minute. After the reactor reached 600° C., a continuous reaction regeneration test was performed. After flowing 100 mL/min of nitrogen into the reactor for 5 minutes, reduction was performed with 50 mL/min of a mixed gas of 50% propane/50% nitrogen for 30 seconds. After flowing nitrogen into the reactor for another 5 minutes, a regeneration process was performed in an atmosphere of air of 100 mL/min for 9 minutes and 30 seconds. Continuous regeneration was performed 1 to 1000 times by regarding this as one reaction regeneration test.

After recovering the catalysts from a continuous reaction regenerator and injecting 0.4 g of the prepared catalysts into the fixed-bed type reactor, it reached up to 600° C., which is a reaction and regeneration temperature, at a temperature raising rate of 10° C. per minute in an atmosphere of a helium gas that is an inert gas. Thereafter, reduction was performed with 105 mL/min of a mixed gas of 50% propane/50% nitrogen for 16 seconds, and a regeneration process was performed in an atmosphere of air of 30 mL/min. Next, oxygen adsorbed on the reactor and catalysts was removed using helium gas for 20 minutes, and then a mixed gas of 50% propane/50% nitrogen was injected at a flow rate of 105 mL/min to perform a reaction at a WHSV 16 h⁻¹. The reaction products were collected every second in a 16-port valve and analyzed through gas chromatography.

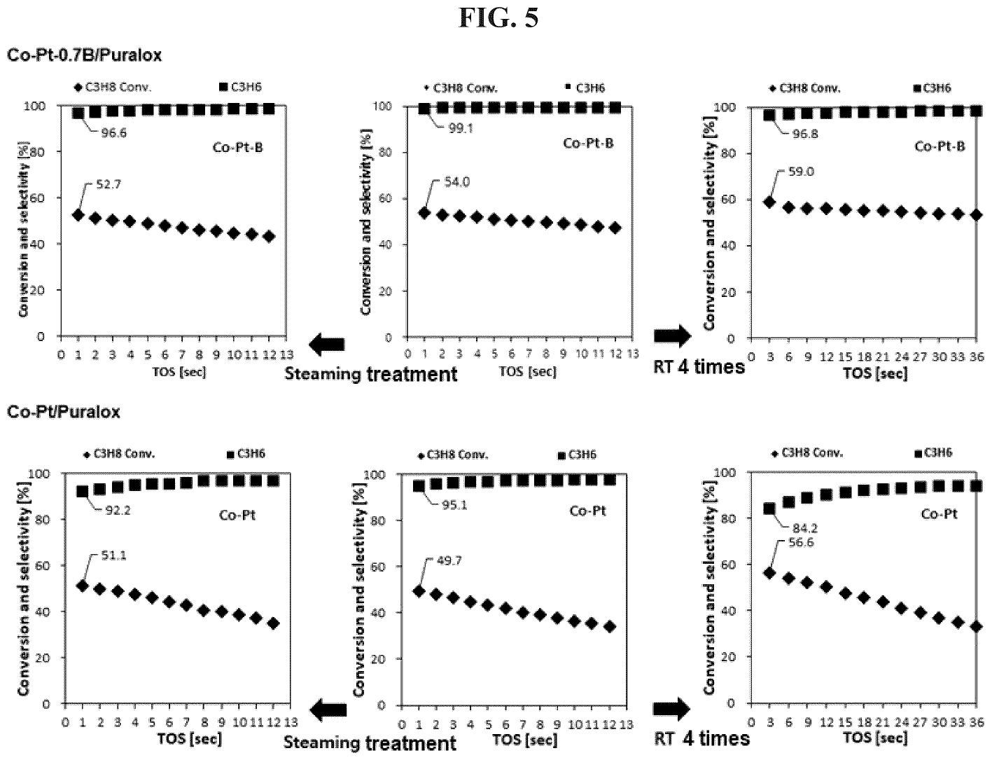

The results of testing the reaction activities of the catalysts prepared above are schematically shown in FIGS. 1 to 8.

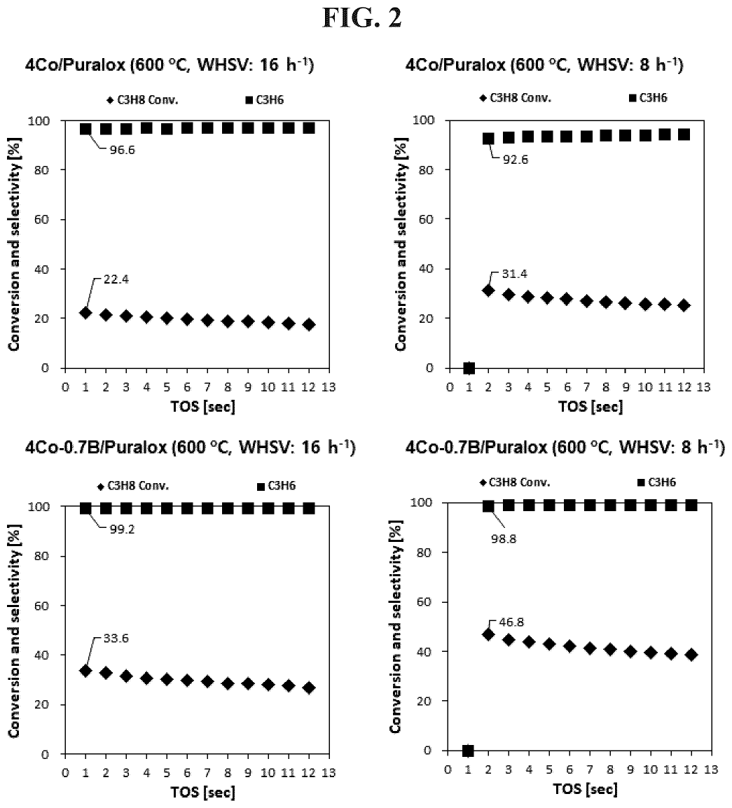

First, as a result of confirming if there is an effect of boron even on Co catalysts except for platinum, as shown in FIG. 2, it could be found that both conversion rate and selectivity were improved. In the case of a 4 wt % Co catalyst, a conversion rate of about 22% and a selectivity of 97% were shown, and when 0.7 wt % of boron was added, the conversion rate increased to 34% and the selectivity showed a further improved activity of 99%. In particular, when the residence time (RT), which is the WHSV 8 h⁻¹ condition, was doubled, the conversion rate increased to 47% and the selectivity was 99%, greatly increasing the improvement effect. As a result, the 4Co-0.7B catalyst, which is a platinum-free catalyst, is also expected to be suitable as a circulating fluidized bed PDH process catalyst through the reaction conditions of the process.

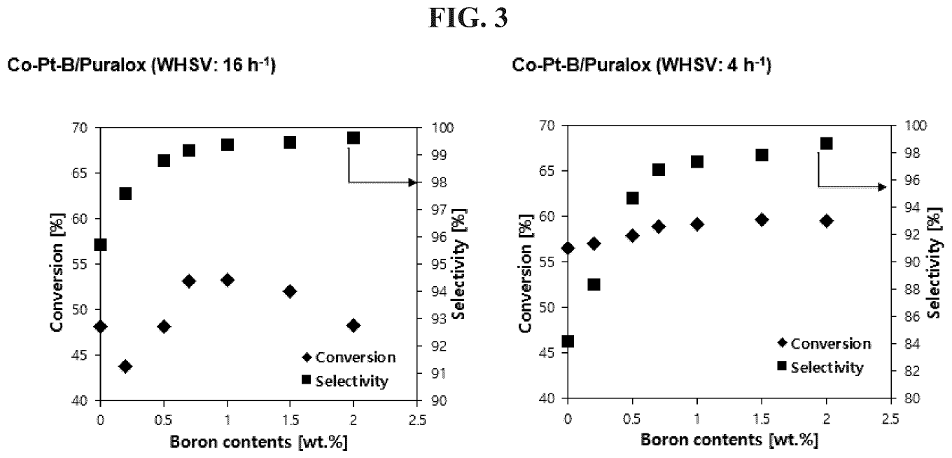

In addition, the effect of a conventionally developed 4Co-0.01Pt/Al₂O₃ catalyst in which 4 wt % of Co and 0.01 wt % of Pt were mixed according to the boron content was tested. A commercially available Puralox alumina carrier was used as the carrier, a catalyst was prepared by increasing the boron content to 0 to 2 wt %, and then PDH initial activity analysis was performed. Reaction tests were conducted under the conditions of a temperature of 600° C. and flow rates of WHSV 16 h⁻¹ and WHSV 4 h⁻¹. FIG. 3 shows average values of conversion rates and selectivities in the initial stage of 1 to 3 seconds of the reaction.

It could be found that propylene selectivity continuously increased as the boron content increased. Under the condition of a flow rate WHSV of 16 h⁻¹, the selectivity of the 4Co-0.01Pt catalyst was about 95%, but the propylene selectivity of 99% or more was shown in the catalyst additionally supported with boron at 0.7 to 2 wt %. When 0.2 wt % of boron was supported, it could be found that the propane conversion rate slightly decreased from 47% to 43%, but increased up to 53% when the boron content was increased to 0.5 to 2 wt %. Afterwards, as a larger amount of boron was supported, the conversion rate slowly decreased.

Meanwhile, under the condition of a flow rate WHSV 4 h⁻¹, the propylene selectivity of the 4Co-0.01Pt catalyst was at a level of about 85%, but the selectivity increased rapidly to 97% as the boron content increased. The propane conversion rate also shown a result of continuously rising slightly.

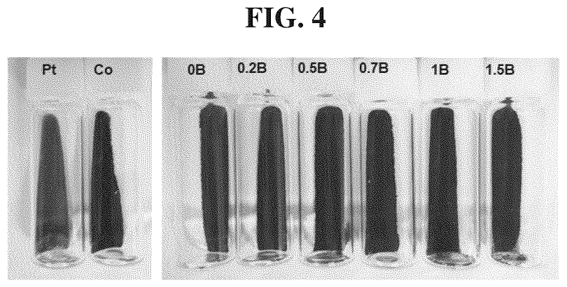

FIG. 4 shows the comparison of the colors of the catalysts after going through the PDH reaction for 1 minute. The Co—Pt catalyst (OB) changed its original cobalt blue color to black due to rapid coke deposition. Looking at the images of the catalysts after the reaction according to the boron content, the original catalyst color, cobalt blue, was maintained as the boron content increased.

In conclusion, looking at the PDH reaction results and the images of the catalysts after use, high selectivity catalysts showing propylene selectivity of about 99% after boron addition were prepared, and accordingly, it could be found that, as the side reaction path was blocked, coke deposition was greatly suppressed.

In addition, the PDH reaction activities of the catalysts with and without the addition of boron were compared. In order to compare the stability after steam treatment and a side reaction increase phenomenon according to the increase in residence time, while carrying out the reaction at the same temperature of 600° C., the results of simultaneously comparing the catalysts after having the flow rates of WHSV 4 h⁻¹ and 16 h⁻¹ and performing steam treatment are shown in FIG. 4.

As a result, after the addition of boron, both conversion rate and selectivity were improved, and in particular, the side reaction blocking effect of boron was confirmed to be remarkable under the WHSV 4 h⁻¹ condition.

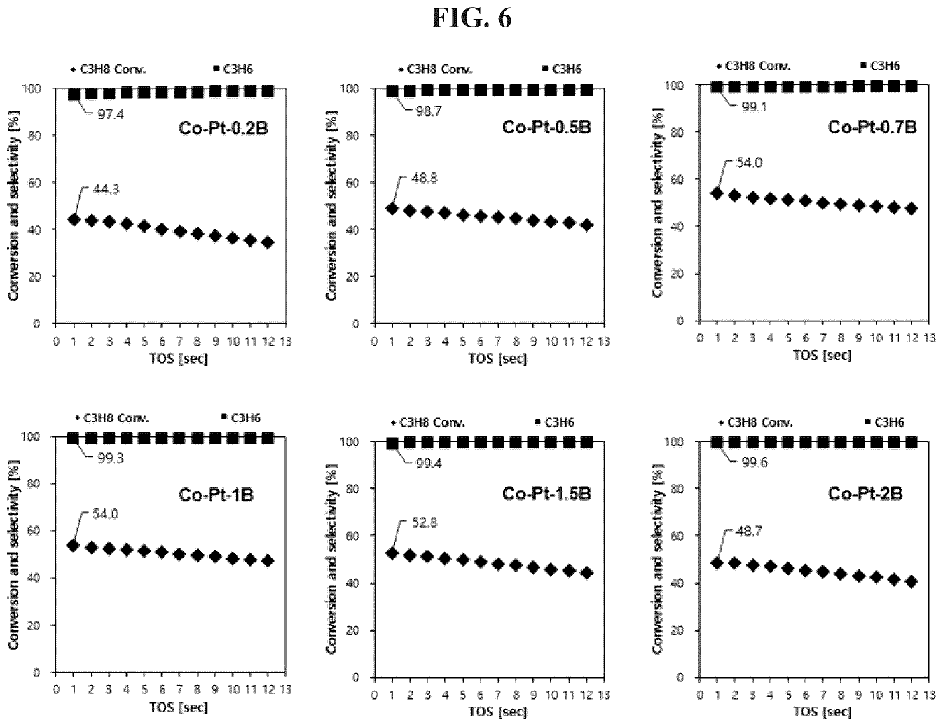

In addition, FIG. 6 shows results of the activity test at the flow rate WHSV 16 h⁻¹ of the Co—Pt—B catalyst according to the boron content. It could be found that the conversion rate and selectivity of all the catalysts containing boron were greatly improved.

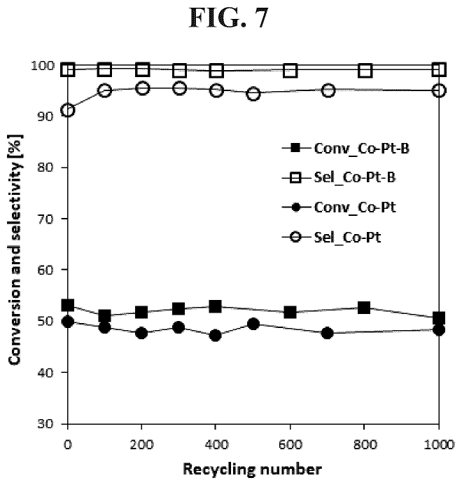

In addition, FIG. 7 shows comparison results of continuous reaction-regeneration and recycling reaction activities of the 4Co-0.01Pt/Al₂O₃ catalyst and the 4Co-0.01Pt-0.7B/Al₂O₃ catalyst. As a result of conducting the recirculation test about 1,000 times, it could be found that there was no problem with catalyst stability even after adding boron, and both conversion rate and selectivity were improved compared to a case before boron was added.

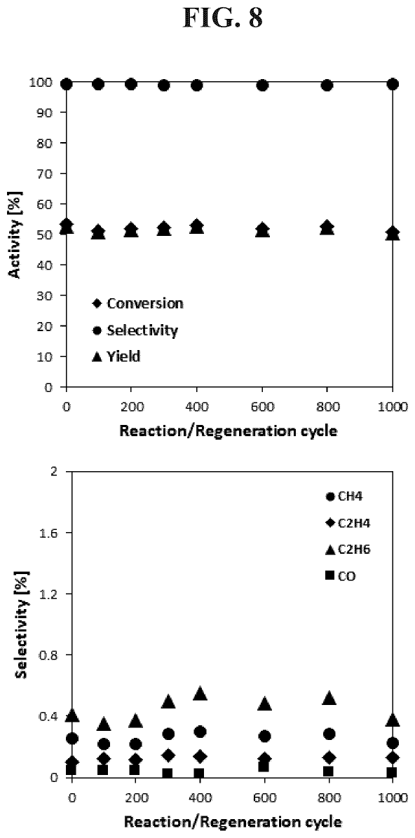

More specifically, as shown in FIG. 8, looking at the detailed results of the continuous reaction-regeneration and recycling reaction activities of the 4Co-0.01Pt-0.7B/Al₂O₃ catalyst, it could be found that the yield was also stable, and the effect of suppressing side reactions was continuously excellent.

This indicates that the effect varies depending on the optimal composition and supported amount of the combined catalyst, even if it is the same dehydrogenation catalytic metal component depending on the reaction process. The amount of platinum required in the FPDH process is an extremely small amount compared to the amount required in the moving bed type process, and the effect is excellent, and the propylene selectivity is also greatly improved due to the introduction of cobalt and boron.

Hereinabove, the embodiments of the present invention have been described in detail, but these embodiments are illustrative and the scope of rights of the present invention is not limited thereto, and it will be apparent to those with ordinary skill in the art that various modifications and variations are possible within the scope without departing from the technical spirit of the present invention described in the claims.

The present invention relates to a catalyst for olefin production having improved selectivity and conversion rate compared to the conventional art in the production of olefins from alkane gases such as ethane, propane, and butane, and a preparation method thereof.

1. A dehydrogenation catalyst for producing olefins from alkane gases, in which a metal active component is supported on an alumina carrier containing boron.

2. The dehydrogenation catalyst of claim 1, wherein the boron is supported in an amount of 0.1 to 2 wt % based on the alumina.

3. The dehydrogenation catalyst of claim 2, wherein the boron is supported in an amount of 0.5 to 2 wt % based on the carrier.

4. The dehydrogenation catalyst of claim 1, wherein the metal active component essentially includes cobalt.

5. The dehydrogenation catalyst of claim 4, wherein the cobalt is supported in an amount of 1 to 5 wt % based on the alumina.

6. The dehydrogenation catalyst of claim 4, wherein the metal active component further includes platinum.

7. The dehydrogenation catalyst of claim 6, wherein the platinum is supported in an amount of 0.001 to 0.05 wt % based on the alumina.

8. A method for preparing a dehydrogenation catalyst for producing olefins from alkane gases, the method comprising the steps of:
impregnating alumina in a boron-containing solution and calcining it to provide a boron-alumina carrier;
providing a solution containing the metal active component;
impregnating the boron-alumina solution in the solution containing the metal active component and drying it; and
calcining the boron-alumina carrier on which the metal active component is supported at 700° C. to 900° C.

9. The method of claim 8, wherein the boron-alumina carrier is calcined at 400 to 600° C.

10. The method of claim 8, wherein the boron is supported in an amount of 0.1 to 2 wt % based on the alumina.

11. The method of claim 10, wherein the boron is supported in an amount of 0.5 to 2 wt % based on the carrier.

12. The method of claim 8, wherein the metal active component essentially includes cobalt.

13. The method of claim 12, wherein the cobalt is supported in an amount of 1 to 5 wt % based on the alumina.

14. The method of claim 12, wherein the metal active component further includes platinum.

15. The method of claim 14, wherein the platinum is supported in an amount of 0.001 to 0.05 wt % based on the alumina.

16. A continuous reaction-regeneration olefin production method including the catalyst of claim 1.

17. The continuous reaction-regeneration olefin production method of claim 16, wherein the reaction temperature is 560 to 640° C.

18. The continuous reaction-regeneration olefin production method of claim 16, wherein alkane, which is a raw material in the olefin production method, has a flow rate (weight hour space velocity) of 4 to 16 h⁻¹.
